(19) United States
(12) Patent Application Publication
(10) Pub. No.: US 2006/0276677 A1
(43) Pub. Date: Dec. 7, 2006

(54) Process for synthesizing alkylated arylamines

(76) Inventors: Hassan Y. Elnagar, Baton Rouge, LA (US); Vincent J. Gatto, Baton Rouge, LA (US); James E. Boone, Baton Rouge, LA (US); Joyce Lo, Lake Jackson, TX (US); Joseph E. Coury, Friendswood, TX (US); Brett Sakahara, Houston, TX (US)

Correspondence Address: EDGAR SPIELMAN;ALBEMARLE CORPORATION, 451 FLORIDA BLVD., BATON ROUGE, LA, 70801, US

(21) Appl. No.: 11/442,856

(22) Filed: May 30, 2006

Related U.S. Application Data
(60) Provisional application No. 60/687,182, filed on Jun. 2, 2005.
(60) Provisional application No. 60/717,322, filed on Sep. 14, 2005.

(51) Int. Cl. C07C 209/24

(52) U.S. Cl. 564/395; 564/409

(57) ABSTRACT

An improved process and novel catalyst system for alkylating arylamines generally comprising the combination of an arylamine and an alkylating agent in the presence of a trialkyl aluminum compound and a hydrogen halide. The improved process and new catalyst system allows for higher total conversion of the arylamine feedstock without sacrificing substitution product selectivity and also allows for the reaction of recycled alkylene feedstock.

### RELATED APPLICATIONS

This application is based on and claims priority to U.S. Provisional Application Ser. Nos. 60/687,182 filed on Jun. 2, 2005 and 60/717,322 filed on Sep. 14, 2005.

### FIELD OF THE INVENTION

The present invention is generally directed towards an improved process for synthesizing alkylated arylamines generally comprising reacting an alkylene, either fresh or a combination of fresh and recycled feedstock, with an arylamine employing either a temperature ramp procedure or milder reaction conditions and utilizing a new catalyst system comprising a trialkyl aluminum compound and a hydrogen halide.

### BACKGROUND

Alkylated arylamines have a variety of different applications. One such application is as an anti-oxidant additive for automotive and industrial lubricants, synthetic, semi-synthetic or natural polymers, in particular thermoplastic plastic materials and elastomers, hydraulic fluids, metal-working fluids, fuels, circulating oils, gear oils and engine oils. In such applications, alkylated arylamines are typically present as an additive having a concentration between about 0.05 wt % and about 2 wt %. Alkylated arylamines contribute to the stabilization of organic materials against oxidative, thermal and/or light-induced degradation. A particular alkylated arylamine, nonylated diphenylamine, is used as an additive for stabilizing organic products that are subject to oxidative degradation. Nonenes are reacted with diphenylamine to synthesize nonylated diphenylamine. Nonenes, sometimes referred to as tripropylene, is a mixture of isomeric C9 olefins. It reacts with diphenylamine to form a mixture of substitution products, namely mono-, di- and tri-alkylated diphenylamine, which remains in solution with any unreacted diphenylamine. Oftentimes, one particular substitution product is desired as is the case with nonylated diphenylamine. The di-alkylated arylamine is desired.

A number of methods of preparing alkylated arylamines are known, most involve reacting alkenes with an arylamine in the presence of a catalyst, attempting to maximize both consumption of the starting material (arylamine) and production of a particular substitution product.

Use of aluminum trichloride as a catalyst in the alkylation of diphenylamine is well established in the art. For example, U.S. Pat. No. 3,496,230 describes production of nonylated diphenylamine (nDPA) using an aluminum trichloride catalyst. See also U.S. Pat. No. 2,776,994 and U.S. Pat. No. 4,739,121. However, aluminum trichloride, because it is a solid, is difficult to handle on an industrial scale.

Similarly, use of clay catalysts in the alkylation of diphenylamine is known in the art. For example, U.S. Pat. No. 6,315,925 describes production of a mixture of nonylated diphenylamines, especially di-nonylated diphenylamines, using acid earth catalysts, particularly acid clays, in the absence of a free protonic acid. See also U.S. Pat. No. 6,204,412 and U.S. Pat. No. 4,824,601. However, use of acid clays as a solid catalyst is generally inefficient, requiring high temperatures.

### SUMMARY OF THE INVENTION

Conventional synthesis routes to alkylated arylamines attempt to maximize high conversion of the arylamine feedstock to the desired substitution product. However, maximizing conversion will often occur at the expense of the desired product selectivity. For example, for alkylated diphenylamine, higher conversion typically results in a higher concentration of the tri-alkylated substitution product. The improved process and novel catalyst system disclosed herein allows for higher total conversion of the arylamine feedstock without sacrificing product selectivity.

In addition to these advantages, the improved process and novel catalyst system also allows for the reaction of recycled alkylene feedstock. Alkylene feeds typically comprise a mixture of isomeric olefins. The position of the double bond in the isomeric olefins determines its reactivity. For example, in a mixture of vinylic (2,2 di-substituted) type and 1,2,3-trisubstituted type olefins, the vinylic olefin is expected to react much faster with the arylamines. Since the alkylene feedstock is charged in excess, the unreacted portion of the alkylene feed will have a higher concentration of the less reactive 1,2,3-trisubstituted type olefins than the fresh feedstock. Thus, when the excess alkylene is collected for recycle, its lower reactivity will require longer reaction times that result in an increase in undesirable substitution products.

The improved process of the present invention generally comprises charging alkylene feed, either an entirely fresh feed or a combination of fresh and recycled alkylenes, and allowing the alkylene feed to react with an arylamine upon the addition of a trialkyl aluminum compound and a hydrogen halide. To maximize total conversion without sacrificing substitution product selectivity for an entirely fresh alkylene feed stock, a milder reaction temperature, a reduced trialkyl aluminum load and excess hydrogen halide are employed. The excess hydrogen halide increases the Lewis acidity of the catalyst system. For an alkylene feed comprising both fresh and recycled alkylenes, similar results are achieved by staging the feed charge. First, the recycled alkylenes are charged at an initially higher reaction temperature using a reduced trialkyl aluminum load and excess hydrogen halide to increase to the Lewis acidity of the catalyst system. The initial charge of recycled alkylenes is followed by the addition of fresh alkylene feed, which is initially allowed to react at the reaction temperature of the initial charge and subsequently reduced to a milder reaction temperature to inhibit undesirable substitution products.

The new catalyst system of the present invention generally comprises the addition to the reaction mass of a trialkyl aluminum compound (Al(alkyl)₃) and a hydrogen halide. Alternatively, sodium halides or similar compounds may be used as a source for the halide, but hydrogen halides are preferred. Suitable trialkyl aluminum compounds include compounds having C₁-C₈ linear or branched alkyl groups that are independently selected (i.e., the alkyl groups of a particular trialkyl aluminum compound need not be the same); however, trialkyl aluminum compounds having C₂-C₄ alkyl groups are preferred due to their ease of handling. The new catalyst system is preferably employed to react alkylene feedstocks having 4-28 carbon atoms.

### DETAILED DESCRIPTION OF THE INVENTION

While the following detailed description generally addresses the alkylation of diphenylamine, it will be known to those skilled in the art that the process and catalyst system described herein may be employed in the alkylation of other arylamines, such as anilines and other similar compounds.

A general reaction scheme for the alkylation of diphenylamine is represented in Scheme 1, showing reaction of diphenylamine with an alkylating agent (alkylene) to yield alkylated diphenylamine upon the addition of a trialkyl aluminum compound and HCl. The catalyst system and processes of the present invention lead to predominant formation of 4,4′dialkyldiphenylamine, with only minor amounts of the ortho-alkylated product. The high degree of para-akylation in the products formed in accordance with the present invention exhibit improved operational performance under conditions of oxidative, thermal, and/or light-induced degradation. In addition to the dialkylated product, small amounts of trialkylated and monoalkylated diphenylamine are formed.

The favoring of the formation of para-isomers is believed to be based on stereo electronic grounds. The active catalytic species formed in the reaction mixture is thought to be one or more chloro-dianilide type structures. The mechanism may be similar to the proposed mechanism for the ortho alkylation of aniline (G. Ecke et al., J. Org. Chem., p639, vol. 22, 1957).

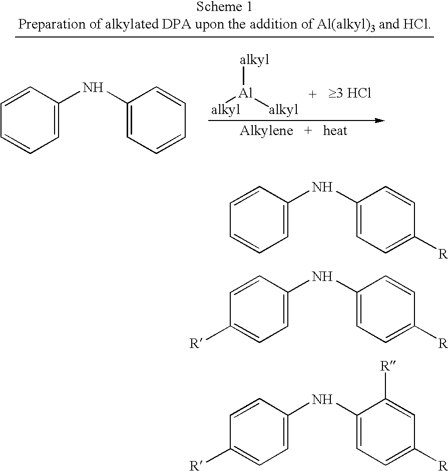

In general, alkylated diphenylamine is prepared by reacting diphenylamine and an alkylating agent (alkylene) upon the addition of a trialkyl aluminum and hydrogen chloride, in which the molar ratio of chloride to aluminum is at least about 3:1 and preferably at least about 4:1. The molar ratio of alkylating agent to diphenylamine can also vary but is preferably between about 2:1 and about 4:1. The molar ratio of Al(alkyl)₃ to diphenylamine can also be varied in the reaction, but preferably ranges from about 0.05:1 to about 0.25:1. R, R′ and R″ may be any linear or branched alkyl group preferably having 4 to 28 carbon atoms corresponding to the olefin isomers of the alkylating agent.

The reactants are preferably allowed to stir at between about 100° C. and 180° C. Diphenylamine conversion of greater than about 95% is observed within about one hour of reaction time at about 150° C. As the concentration of the di-alkylated product increases, the reaction to the tri-alkylated product competes more effectively with the depleted diphenylamine and becomes especially effective with time and/or elevated temperatures.

As stated above, when employing an alkylating agent comprising both fresh and recycled alkylene, the recycled alkylene has a much lower reactivity and tends to produce a greater amount of undesirable substitution products due to the longer reaction times and/or temperatures necessary for high total conversion. Thus, to ensure that proper product specifications are maintained, the recycled alkylenes are preferably limited to about 40% of the total alkylene feed. The recycled alkylenes are allowed to react with the diphenylamine before addition of the fresh alkylenes, this way the aromatic ring is forced to react with the less reactive olefin.

One preferred embodiment of the catalyst system is obtained by adding a trialkyl aluminum compound and gaseous HCl to diphenylamine. The gaseous HCl is bubbled through the trialkyl aluminum compound and diphenylamine mixture creating an exotherm. In effect, mixed alkyl chloride catalyst derivatives are generated in-situ comprising one or more of the following species: AlCl₃, Al(alkyl)Cl₂, Al(alkyl)₂Cl, Al₂(alkyl)₂Cl₄, [Al(alkyl)Cl₃]⁻, [Al₂(alkyl)₂Cl₅]⁻, [Al₃(alkyl)₃Cl₇]⁻, and [Al₂(alkyl)Cl₆]⁻. The presence of the ionic species accelerates reaction rate by enhancing Lewis acidity, particularly in the presence of excess HCl. Because the above-listed species are important in the reaction mechanism, mono- and/or dialkyl/halide aluminum compounds may be employed as an alternative to trialkyl aluminum compounds in the catalyst system.

### EXAMPLES

### Example 1

The following general procedure was employed to preparation nonylated diphenylamine.

The reaction glassware was purged with nitrogen before use and the reaction was run under nitrogen. The general molar feed feed ratios are: C₉:DPA=2.89; TEA:DPA=0.157; Cl:Al (catalyst)=˜3.3-3.5.

To a 500 mL boiling flask, 85 g diphenylamine (DPA) was added. The flask was purged with nitrogen for 5 minutes and the flask heated to 60° C. to melt the DPA. To an addition column attached to the flask, 183 g of nonenes (C₉) was added. Using appropriate precautions and transfer techniques, 9 g triethylaluminum (TEA) was transferred to the reaction flask, followed immediately by addition of the nonenes from the addition column. After vigorous stirring, the targeted amount of HCl(g) was bubbled through the reaction mixture in the vessel. The reaction was heated at 150° C. for 3 hrs, with samples taken at t=0, 1.5 and 3 hours. The reactor was then cooled and the crude product decanted and weighed.

Examples 1A-1L follow the General Procedure using TEA+HCl as the catalyst system with the noted variations in reactant quantities and reaction times summarized in Table 1. Each reaction was run at 150° C. under slightly positive nitrogen pressure.

TABLE 1
Exemplary preparation of alkylated diphenylamine and product distribution.

| | | 1A | 1B | 1C | 1D | 1E | 1F | 1G | 1H | 1I | 1J | 1K | 1L |
|---|---|---|---|---|---|---|---|---|---|---|---|---|---|
| Sample | | 1/23 | 1/28 | 2/3 | 2/4 | 2/9 | 2/10 | 2/13 | 2/14 | 2/16 | 2/18 | 4/20 | 4/21 |
| React. | Time | 21.0 | 9.0 | 3.0 | 3.0 | 3.0 | 2.5 | 3.0 | 3.0 | 3.0 | 3.0 | 3.3 | 3.5 |
| Cond. | (hrs) | | | | | | | | | | | | |
| | Wt % Al | 0.79 | 0.92 | 1.23 | 1.00 | 1.44 | 1.18 | 1.33 | 1.32 | 1.30 | 1.18 | 1.41 | 1.38 |
| | Wt % Cl | 3.17 | 3.49 | 5.34 | 4.23 | 5.60 | 5.2 | 5.69 | 6.26 | 5.90 | 5.59 | 6.20 | 6.44 |
| | Cl:Al | 3.1 | 2.9 | 3.3 | 3.2 | 3.0 | 3.4 | 3.3 | 3.6 | 3.5 | 3.6 | 3.3 | 3.6 |
| End | DPA | | 3.2 | 1.4 | 1.4 | 1.3 | 1.3 | 1.0 | 0.9 | 1.0 | 0.9 | 1.6 | 1.2 |
| React. | MONO | | 27.9 | 21.4 | 21.7 | 21.3 | 21.3 | 19.1 | 18.4 | 18.3 | 18.2 | 21.2 | 19.9 |
| Comp. | DI | | 64.6 | 71.1 | 71.6 | 70.8 | 71.0 | 72.1 | 72.2 | 72.4 | 72.8 | 71.7 | 71.0 |
| (area %) | TRI | | 4.2 | 6.1 | 6.3 | 6.5 | 6.4 | 7.8 | 8.5 | 8.4 | 8.0 | 5.2 | 7.7 |
| Final | C9 | 34.8 | 3.8 | 1.8 | 1.9 | 0.2 | 0.3 | 0.2 | 0.2 | 0.3 | 0.3 | 0.7 | 0.3 |
| Prod | DPA | 0.8 | 3.1 | 1.2 | 1.3 | 1.2 | 1.2 | 0.9 | 0.9 | 0.9 | 0.8 | 1.4 | 1.1 |
| Comp. | MONO | 12.0 | 26.8 | 20.0 | 20.6 | 20.0 | 20.3 | 18.5 | 17.8 | 17.5 | 16.4 | 20.7 | 18.8 |
| (area %) | DI | 47.3 | 62.1 | 70.1 | 69.7 | 71.2 | 71.0 | 72.0 | 71.7 | 71.9 | 73.4 | 70.4 | 71.1 |
| | TRI | 4.6 | 4.1 | 6.7 | 6.3 | 7.1 | 7.0 | 8.2 | 9.1 | 9.0 | 8.8 | 5.6 | 8.5 |

| | | | | | | | | | | | | |
|---|---|---|---|---|---|---|---|---|---|---|---|---|
| Viscosity (cSt) | n/a | 220 | 315 | 300 | 483 | 482 | n/a | n/a | n/a | n/a | n/a | 529 |
| Color (Gardner) | n/a | n/a | n/a | n/a | 3.2 | n/a | n/a | n/a | n/a | n/a | n/a | 3.8 |

MONO = monononylated diphenylamine
DI = dinonylated diphenylamine
TRI = trinonylated diphenylamine
DPA = diphenylamine
C9 = nonenes

### Example 2

In a dry box, TEA (10 g, 0.088 mol) was charged into 1-1 round bottom flask containing a mixture of 36.0 g (0.28 mol, ˜20% of total required nonenes) of recycled nonenes and 42.0 g (0.33 mol) fresh olefin (total 78 g, ˜0.62 mol). The flask was transferred into a hood and DPA (85.0 g, 0.50 mol) was quickly added and stirred while bubbling HCl under a nitrogen atmosphere. The reactor was equipped with stirring bar, thermocouple and was connected to cooling condenser.

Approximately 30 g HCl (0.82 mol, Cl/Al ratio ˜9.3) was charged over 10 min and an exotherm (136° C.) was observed. Heating was set at 150° C. When reaction temperature reached 150° C., GC analysis indicated ˜67% conversion of DPA to a mixture of mostly mono-nonylated material. No tri-alkylated product was formed.

The balance of the required 2.9 equivalent of nonenes (105 g fresh olefins, 0.83 mol, ˜183 g total charged nonenes, ˜2.9 equivalents) was then added over 17 min while heating at 150° C. After 1 h of adding all nonenes, GC analysis showed ˜98% conversion of the DPA to products. After 2 h of heating, the DPA conversion slightly increased to ˜98.4%, and heating was discontinued.

The reaction mixture was quenched by pouring over 150 g of 25 wt. % caustic solution. The organic phase was separated after shaking vigorously with the aqueous solution and then was transferred into a 1-1 round bottom flask connected to a receiver and equipped with a thermocouple and magnetic stirring bar. The crude mixture was heated gradually for about 0.5 h (150° C.) by means of a heating mantle under vacuum to remove the excess nonenes and the residual water. About 56 g of dried nonenes (MgSO4) was collected in the dry ice cooled receiver.

The NDPA was filtered under vacuum while hot over 20 g of active basic aluminum oxide bed to obtain 172 g of NDPA as a light brown oil. Nitrogen analysis of NDPA (nonylated diphenyamine) was determined to be 3.86% by weight.

The isolated product was analyzed by GC. The product distribution in Table 2 shows the high-degree of para-alkylation when the catalyst system and processes of the present invention are employed.

TABLE 2
GC Analysis of Isolated product

| Components | Area % |
|---|---|
| DPA | 1.53 |
| o-mono-alkylated DPA | 0.28 |
| p-mono-alkylated DPA | 21.91 |
| o-di-alkylated DPA | 3.05 |
| p-di-alkylated DPA | 65.35 |
| tri-alkylated DPA | 7.70 |

### Example 3

TEA (7.0 g, 61 mmol) was charged into 1-1 round bottom flask (equipped with magnetic stirrer, thermocouple, and cooling condenser) containing 120 g (0.95 mol) nonenes. Solid DPA (85 g, 0.50 mol) was added to the nonene/TEA mixture and the slurry was stirred while bubbling HCl under a nitrogen atmosphere.

Approximately 11.7 g HCl (0.32 mol, Cl/Al ratio ˜5.2) was charged over 15 min and heating temperature was set at 125° C. GC analysis indicated 88% DPA conversion to products in less than 2 hours of heating. The third equivalent of nonenes was added (61 g, total 181 g) and the reaction progress was monitored and summarized as shown Table 3. A total of fifteen hours of heating, after addition of all nonenes, was necessary for >99% DPA conversion.

The crude reaction mass was poured slowly over 125 g of 25 wt. % caustic solution, in a separate 1-L round bottom flask equipped with mechanical stirrer and was vigorously mixed (320 rpm, 25 min) and the two phases were allowed to separate (30 min).

The organic phase was transferred into a 1-1 round bottom flask equipped with a magnetic stirrer, and a short condenser connected to dry-ice cooled receiver. The light brown reaction mass was heated (heating mantle) gradually to 150° C. under 15 mm Hg vacuum for about 0.5 h to remove the excess nonenes and the residual water. Fourty three (43) grams of dried (MgSO4) nonenes were collected.

TABLE 3
Reaction progress (GC area %) @ 125° C.

| Reaction time | % DPA |
|---|---|
| (h) | Conversion |
| 1 | 95.7 |
| 4 | 97.9 |
| 7 | 98.0 |
| 15 | 99.2 |

The NDPA was filtered under vacuum while hot (130° C.) over active basic aluminum oxide (20 g) to remove trace solid salts. The isolated NDPA (179 g) was analyzed by GC, the results of which are shown in Table 4.

TABLE 4
GC Area % Analysis of NDPA

| Components | GC Area % |
|---|---|
| DPA | 0.72 |
| mono-alk-DPA | 15.8 |
| di-alk-DPA | 77.9 |
| tri-alk-DPA | 5.4 |

### Example 4

TEA (7.0 g, 61 mmol) was charged into 1-1 round bottom flask containing 70.0 g (0.55 mol) distilled recycled nonenes. DPA (85 g, 0.50 mol) was added and the slurry was stirred under a nitrogen atmosphere. The reactor was equipped with stirring bar, thermocouple and was connected to a cooling condenser.

Approximately 17.0 g HCl (0.466 mol, Cl/Al ratio ˜7.6) was bubbled into the slurry over 22 min and an exotherm (101° C.) was observed. Heating was initially set at 150° C. for 0.5 h to insure recycled olefin reaction. Addition of fresh nonenes (113 g, total of 183 g olefins) was then followed over 14 min to the gently refluxing reaction mixture. GC analysis indicates 92.1% DPA conversion immediately at the end of nonenes addition.

Heating was immediately set at 125° C. and the reaction progress was monitored by GC, as in the above example, the results of which are shown in Table 5. A total of fifteen hours of heating after addition of all nonenes was necessary for greater than 99% DPA conversion.

TABLE 5
Reaction Progress (GC area %)

| Reaction time | % DPA |
|---|---|
| (h) | Conversion |
| 0 | 92.1 |
| 1 | 95.9 |
| 3 | ˜98 |
| 7 | 98.3 |
| 15 | ˜99.3 |

The crude reaction mass was poured slowly over 125 g of 25 wt. % caustic solution, in a separate 1-L round bottom flask equipped with mechanical stirrer and was vigorously mixed (320 rpm, 40 min). The two phases were allowed to stand 30 min before separation.

The organic phase was transferred into a 1-1 round bottom flask equipped with a magnetic stirrer, and a short condenser connected to dry-ice cooled receiver. The reaction mass was heated gradually to 150° C. under 12 mm Hg vacuum for about 0.5 h to remove the excess nonenes and the residual water. Forty three (43) grams of dried (MgSO4) nonenes were collected.

The NDPA was filtered under vacuum while hot (125° C.) over active basic aluminum oxide (20 g) to remove trace solid salts. The isolated NDPA (182 g) was analyzed by GC and the data shown in Table 6 below.

TABLE 6
GC Area % Analysis of NDPA

| Components | GC Area % |
|---|---|
| DPA | 0.68 |
| mono-alk-DPA | 15.7 |
| di-alk-DPA | 75.2 |
| tri-alk-DPA | 8.3 |

### Example 5

TEA (10.0 g, 61 mmol) was charged into 1-1 round bottom flask (equipped with a magnetic stirrer, a thermocouple, and a cooling condenser) and contained 61 g (0.48 mol) nonenes. DPA (85 g, 0.50 mol) was added to the nonene/TEA mixture and stirred while bubbling gaseous HCl intermittently under a nitrogen atmosphere.

Approximately 11.9 g HCl (0.32 mol, Cl/Al ratio ˜3.7) was initially charged over 30 min. Heating temperature was set initially at 150° C. and heated for about 11 min at that temperature. A second equivalent of nonenes (61 g, total 122 g) was added over 10 min and heating continued for 1 h at 150° C. The total HCl added at this point was 13.5 g, (Cl/Al ˜4.2). GC analysis of the crude reaction mixture indicated ˜94% DPA conversion. The third nonenes portion (61 g, total 183 g) was added quickly and temperature was reset at 140° C. and heated for 1 h. GC analysis indicated ˜98.1% conversion with formation of minor amounts of tri-alkylated DPA. Heating continued for a second hour at 140° C. while bubbling an additional 2.1 g HCl (total 15.6, Cl/Al 4.9) and the DPA conversion increased to 98.6%.

The fourth and last nonenes portion was added (61 g, total 244 g, ˜3.86 equivalents) over 8 min. The reaction temperature was reset at 130° C. and heated for about two hours to exceed 99% conversion (less than 6 h of heating).

The crude reaction mass was poured over 125 g of 25 wt. % caustic solution, in a separate 1-L round bottom flask equipped with mechanical stirrer and was vigorously mixed (320 rpm, 30 min). The two phases were allowed to separate. The organic phase was transferred into a 1-1 round bottom flask equipped with a magnetic stirrer, and a short condenser connected to dry-ice cooled receiver.

The brown reaction mass was heated (heating mantle) gradually to 150° C. under 11 mm Hg vacuum for about 0.5 h to remove the excess nonenes and the residual water.

The crude NDPA was filtered under vacuum while hot (85° C.) over active basic aluminum oxide (20 g) to remove trace solid salts. The isolated NDPA (178 g) was analyzed by GC. The DPA concentration was 0.49 wt. % and the tri-alkylated-DPA concentration was 9.56%.

### Example 6

85 g of diphenylamine (DPA, 0.50 mol), 210 g of propylene tetramer (C12 olefins) (˜1.25 mol), 80 ml of 1.0 M TEA solution in heptane (0.08 mol) was charged into a three neck flask under nitrogen atmosphere. HCl gas (6 g, 0.16 mol) was bubbled into the mixture and the reaction mass was heated for 4 hours at 150° C. Approximately 90% DPA conversion was determined by GC analysis. Additional HCl (4 g, 0.11 mol) was bubbled and the reaction mass was heated for an additional 3 hours. GC analysis indicated about 94% DPA conversion. 40 g of excess propylene tetramer was added and the mixture was heated for 8 hours. Approximately 3% of unreacted DPA persisted in the reaction mixture.

The reaction mass was quenched by pouring the mass over a 25% aqueous NaOH solution and then washed with water (3×400 ml). The organic phase was heated to remove moisture, heptane and and excess olefin by heating gradually to 180° C. under reduced pressure to obtain 219 g of thick brown oil.

The DPA was mostly removed by purging the heated oil (150° C.) with steam under vacuum by a slow subsurface feeding of water (0.2 liter) to the heated oil at a rate of 0.5 ml/min using Masterflex feeding pump. The DPA was collected with the condensed steam in a dry ice cooled receiving flask. The propylene tetramer-DPA was analyzed by GC and the data is shown in Table 7 below.

TABLE 7
GC Area % Analysis of propylene tetramer-DPA

| Components | GC Area % |
|---|---|
| DPA | <0.1 |
| mono-alk-DPA | 21.35 |
| di-alk-DPA | 66.74 |
| tri-alk-DPA | 11.88 |

### Example 7

85 g of diphenylamine (DPA, 0.50 mol), 217 g propylene tetramer, a mixture of Et₂AlCl (50 mL of 1.0 M solution in heptane, 0.05 mol) and AlCl₃ (7.0 g, 0.05 mol) was charged to a three neck flask under nitrogen atmosphere. No product was detected when the reaction mixture was heated for two hours. HCl gas (total of 14 g, 0.38 mol) was bubbled into the mixture and the reaction mass was heated for a total of 9 hours at 150° C. After caustic workup and removal of the excess olefin under vacuum, the resulting oil was filtered over a celite. The results of the GC analysis of the resulting brown oil are shown in Table 8.

TABLE 8
GC Area % Analysis of propylene tetramer-DPA

| Components | GC Area % |
|---|---|
| DPA | 2.86 |
| mono-alk-DPA | 29.78 |
| di-alk-DPA | 59.17 |
| tri-alk-DPA | 6.14 |

The foregoing examples are not limiting and are merely illustrative of various aspects and embodiments of the present invention. One skilled in the art will readily appreciate that the present invention is well adapted to carry out the objects and obtain the ends and advantages mentioned, as well as those inherent therein. Certain modifications and other uses will occur to those skilled in the art, and are encompassed within the spirit of the invention, as defined by the scope of the claims.

What is claimed is:

1. A method for alkylating arylamine comprising the steps of: creating a reaction mass from materials comprising arylamine, alkylating agent, trialkyl aluminum and hydrogen halide; and forming alkylated arylamine.

2. The method of claim 1, wherein the molar ratio of halide to aluminum is at least about 3:1.

3. The method of claim 1, wherein the molar ratio of alkylating agent to arylamine is between about 2:1 to about 4:1.

4. The method of claim 1, wherein the molar ratio of trialkyl aluminum to arylamine is between about 0.05:1 to about 0.25:1.

5. The method of claim 1, wherein the arylamine is diphenylamine.

6. The method of claim 1, wherein the alkylating agent is an alkylene having 4 to 28 carbon atoms.

7. The method of claim 1, wherein the alkylating agent is a mixture of isomeric olefins having 4 to 28 carbon atoms.

8. The method of claim 7, wherein the alkylating agent is an isomeric mixture of nonenes.

9. The method of claim 8, wherein the arylamine is diphenylamine.

10. The method of claim 1, wherein the alkyl groups of the trialkyl aluminum comprise 1 to 8 carbon atoms.

11. The method of claim 10, wherein the trialkyl aluminum is triethyl aluminum.

12. The method of claim 1, wherein the hydrogen halide is hydrogen chloride.

13. A process for alkylating arylamine comprising the steps of:
creating a reaction mass from materials comprising arylamine, alkylating agent, trialkyl aluminum and hydrogen halide;
allowing the reaction mass to blend for at least about one hour while heating to a temperature between about 100° C. to about 180° C.;
removing essentially all of the unreacted alkylating agent; and
isolating the resulting alkylated arylamine.

14. The process of claim 13, wherein the alkylating agent is fresh, unreacted alkylene.

15. The process of claim 14, wherein the trialkyl aluminum and a portion of the total amount of alkylating agent are initially combined followed by the addition of arylamine and subsequent bubbling of hydrogen halide through the reaction mass.

16. The process of claim 14, wherein the arylamine is combined with alkylating agent and the trialkyl aluminum followed by bubbling of hydrogen halide through the reaction mass.

17. The process of claim 14, wherein the reaction mass is heated to an initial reaction temperature and subsequently cooled to a lower reaction temperature.

18. The process of claim 17, wherein the reaction mass is heated to an initial reaction temperature between about 140° C. and about 180°.

19. The process of claim 18, wherein the reaction mass is subsequently cooled to a lower reaction temperature between about 120° C. and about 140° C.

20. The process of claim 19, wherein the arylamine is diphenylamine.

21. The process of claim 20, wherein the alkylating agent is an isomeric mixture of nonenes.

22. The process of claim 13, wherein the alkyl groups of the trialkyl aluminum comprise 1 to 8 carbon atoms.

23. The process of claim 22, wherein the trialkyl aluminum is triethyl aluminum.

24. The process of claim 13, wherein the hydrogen halide is hydrogen chloride.

25. The process of claim 13, wherein the alkylating agent comprises fresh, unreacted alkylene and recycled alkylene.

26. The process of claim 25, wherein the trialkyl aluminum and a portion of the total amount of alkylating agent are initially combined followed by the addition of arylamine and subsequent bubbling of hydrogen halide through the reaction mass.

27. The process of claim 25, wherein the alkylating agent and the trialkyl aluminum are combined with arylamine followed by bubbling of hydrogen halide through the reaction mass.

28. The process of claim 26, wherein the portion of the total amount of alkylating agent initially combined comprises recycled alkylene.

29. The process of claim 28, wherein the reaction mass is heated to an initial reaction temperature upon the combining of recycled alkylene.

30. The process of claim 29, wherein the reaction mass is heated to an initial reaction temperature between about 140° C. and about 180° C.

31. The process of claim 29, wherein the fresh, unreacted alkylene is combined to the reaction mass subsequent to its heating to an initial reaction temperature.

32. The process of claim 31, wherein the reaction mass is cooled to a lower reaction temperature upon the combining of the fresh, unreacted alkylene.

33. The process of claim 32, wherein the reaction mass is cooled to a lower reaction temperature between about 120° C. and about 140° C.

34. A composition for catalyzing the alkylation of arylamine with alkylating agent in a reaction mass, the composition comprising trialkyl aluminum and hydrogen halide.

35. The composition of claim 34, wherein the molar ratio of halide to aluminum is at least about 3:1.

36. The composition of claim 34, wherein the molar ratio of trialkyl aluminum to arylamine is between about 0.05:1 to about 0.25:1.

37. The composition of claim 34, wherein the arylamine is diphenylamine.

38. The composition of claim 34, wherein the alkyl groups of the trialkyl aluminum comprise 1 to 8 carbon atoms.

39. The composition of claim 38, wherein the trialkyl aluminum is triethyl aluminum.

40. The composition of claim 34, wherein the hydrogen halide is hydrogen chloride.

41. An alkylated arylamine mixture produced by creating a reaction mass from materials comprising arylamine, alkylating agent, trialkyl aluminum and hydrogen halide, allowing the reaction mass to blend for at least about one hour while heating to a temperature between about 100° C. to about 180° C., removing essentially all of the unreacted alkylating agent; and isolating the resulting alkylated arylamine mixture, the alkylated arylamine mixture comprising mono-, di-, and tri-alkylated arylamine components, wherein the alkyl group of the mono-alkylated arylamine component is predominantly in the 4position and the alkyl groups of the di-alkylated arylamine component is predominantly in the 4,4′-position.

42. The alkylated arylamine mixture of claim 41, wherein the molar ratio of halide to aluminum in the reaction mass is at least about 3:1.

43. The alkylated arylamine mixture of claim 41, wherein the molar ratio of alkylating agent to arylamine in the reaction mass is between about 2:1 to about 4:1.

44. The alkylated arylamine mixture of claim 41, wherein the molar ratio of trialkyl aluminum to arylamine in the reaction mass is between about 0.05:1 to about 0.25:1.

45. The alkylated arylamine mixture of claim 41, wherein the arylamine is diphenylamine.

46. The alkylated arylamine mixture of claim 41, wherein the alkylating agent is an alkylene having 4 to 28 carbon atoms.

47. The alkylated arylamine mixture of claim 41, wherein the alkylating agent is a mixture of isomeric olefins having 4 to 28 carbon atoms.

48. The alkylated arylamine mixture of claim 41, wherein the alkylating agent is an isomeric mixture of nonenes.

49. The alkylated arylamine mixture of claim 48, wherein the arylamine is diphenylamine.

50. A method of stabilizing a fluid subject to oxidative, thermal and/or light-induced degradation comprising the step of adding to the fluid a suitable amount of an alkylated arylamine mixture produced by creating a reaction mass from materials comprising arylamine, alkylating agent, trialkyl aluminum and hydrogen halide, allowing the reaction mass to blend for at least about one hour while heating to a temperature between about 100° C. to about 180° C., removing essentially all of the unreacted alkylating agent, and isolating the resulting alkylated arylamine mixture, the alkylated arylamine mixture comprising mono-, di-, and tri-alkylated arylamine components, wherein the alkyl group of the mono-alkylated arylamine component is predominantly in the 4position and the alkyl groups of the di-alkylated arylamine component is predominantly in the 4,4′-position.

51. The method of claim 50, wherein the arylamine is diphenylamine.

52. The alkylated arylamine mixture of claim 50, wherein the alkylating agent is an alkylene having 4 to 28 carbon atoms.

53. The alkylated arylamine mixture of claim 50, wherein the alkylating agent is a mixture of isomeric olefins having 4 to 28 carbon atoms.

54. The alkylated arylamine mixture of claim 50, wherein the alkylating agent is an isomeric mixture of nonenes.

55. The alkylated arylamine mixture of claim 54, wherein the arylamine is diphenylamine.

56. A composition comprising a fluid subject to oxidative, thermal and/or light-induced degradation and an alkylated arylamine mixture produced by creating a reaction mass from materials comprising arylamine, alkylating agent, trialkyl aluminum and hydrogen halide, allowing the reaction mass to blend for at least about one hour while heating to a temperature between about 100° C. to about 180° C., removing essentially all of the unreacted alkylating agent; and isolating the resulting alkylated arylamine mixture, the alkylated arylamine mixture comprising mono-, di-, and tri-alkylated arylamine components, wherein the alkyl group of the mono-alkylated arylamine component is predominantly in the 4position and the alkyl groups of the di-alkylated arylamine component is predominantly in the 4,4′-position.

57. The composition of claim 56, wherein the arylamine is diphenylamine.

58. The composition of claim 56, wherein the alkylating agent is an alkylene having 4 to 28 carbon atoms.

59. The composition of claim 56, wherein the alkylating agent is a mixture of isomeric olefins having 4 to 28 carbon atoms.

60. The composition of claim 56, wherein the alkylating agent is an isomeric mixture of nonenes.

61. The composition of claim 60, wherein the arylamine is diphenylamine.

62. A method for nonylating diphenylamine comprising the steps of: creating a reaction mass from materials comprising diphenylamine, nonenes, triethyl aluminum and hydrogen chloride, wherein the molar ratio of nonenes to diphenylamine is between about 2:1 to about 4:1, the molar ratio of triethyl aluminum to diphenylamine is between about 0.05:1 to about 0.25:1 and the molar ratio of chloride to aluminum is at least about 3:1; and forming nonylated diphenylamine.

63. The method of claim 62, wherein the nonenes are fresh nonenes.

64. The method of claim 62, wherein the nonenes comprise fresh and recycled nonenes.

65. A process for nonylating diphenylamine comprising the steps of:
creating a reaction mass from materials comprising diphenylamine, recycled nonenes, triethyl aluminum and hydrogen chloride, wherein the molar ratio of triethyl aluminum to diphenylamine is between about 0.05:1 to about 0.25:1 and the molar ratio of chloride to aluminum is at least about 3:1;
allowing the reaction mass to blend for at least about one hour while heating to an initial reaction temperature between about 140° C. to about 180° C.;
adding fresh nonenes to the reaction mass, such that the molar ratio of total nonenes to diphenylamine is between about 2:1 to about 4:1, and blending the reaction mass for at least about one hour while reducing the reaction temperature to between about 120° C. to about 140° C.;
removing essentially all of the unreacted nonenes; and
isolating the resulting nonylated diphenylamine.
